(19) United States
(12) Patent Application Publication
(10) Pub. No.: US 2014/0010852 A1
(43) Pub. Date: Jan. 9, 2014

(54) COSMETIC TREATMENT METHOD COMPRISING THE APPLICATION OF A COATING BASED ON AN AEROGEL COMPOSITION OF LOW BULK DENSITY

(71) Applicant: Henri Samain, Bievres (FR)

(72) Inventor: Henri Samain, Bievres (FR)

(73) Assignee: L'OREAL, Paris (FR)

(21) Appl. No.: 13/980,388

(22) PCT Filed: Jan. 20, 2012
(86) PCT No.: PCT/FR12/50128

§ 371 Date: Sep. 26, 2013

Related U.S. Application Data
(60) Provisional application No. 61/448,313, filed on Mar. 2, 2011.
(60) Provisional application No. 61/448,315, filed on Mar. 2, 2011.

(30) Foreign Application Priority Data
Jan. 20, 2011 (FR) 1150467
Jan. 20, 2011 (FR) 1150468

(51) Int. Cl. A61K 8/04; A61Q 5/00; A61K 8/19; A61K 8/58

(52) U.S. Cl. CPC A61K 8/042; A61K 8/585; A61Q 5/00; A61K 8/19
USPC 424/401; 424/70.1

(57) ABSTRACT

The present invention relates to a cosmetic treatment method comprising the formation of a coating on keratin fibres characterized in that it comprises: 1) the preparation of an aerogel precursor composition comprising:—at least one organic solvent chosen from acetone, C₁-C₄ alcohols, C₁-C₆ alkanes, C₁-C₄ ethers, which may or may not be perfluorinated, and mixtures thereof and at least one precursor compound that contains:—at least one atom chosen from silicon, titanium, aluminium and zirconium,—at least one hydroxyl or alkoxy function directly attached to the atom chosen from silicon, titanium, aluminium and zirconium by an oxygen atom, and,—optionally an organic group directly attached to the atom chosen from silicon, titanium, aluminium and zirconium by a carbon atom, 2) the removal of the solvent or solvents resulting in the formation of an aerogel composition having a bulk density less than or equal to 0.35 g/cm³, 3) the application to the keratin fibres of the aerogel composition resulting from step 2) or of the aerogel precursor composition resulting from step 1). Advantageously, the molar ratio between the precursor compounds and the solvent is at most 1/20.

The present invention relates to a cosmetic treatment method comprising the formation of a coating on keratin fibers, on the basis of an aerogel composition with a low bulk density. The aerogel composition is formed either in situ, in other words directly on the keratin fibers, or ex situ, in which case, once it has been formed, it is applied to the keratin fibers.

There are numerous cosmetic applications where the aim is to cover keratinous materials with a coating or film that has mechanical and cosmetic characteristics that are specific to the intended use. A particular aim is to be able to produce coatings on the hair that are flexible and readily deformable and that, after drying, produce surface effects such as hold, covering, coloring, and shine effects; protective effects against radiation, pollutants, water, or humidity; modification of the behavior of wet hair, in particular a modification of the feel of hair that has suffered damage; biological effects; or a reduction in defects.

Generally speaking, coatings are used that are based on fatty or waxy substances, or polymeric coatings. These solutions are effective but do have certain drawbacks, in particular a heaviness if the coating is thick. This drawback is especially problematic for hair applications.

In order to solve these problems, one proposed solution to consist in maximally streamlining the thickness of the coating. This solution is sometimes effective, but remains limited to the following situations:
protection from cold or heat;
absorption of body fluids;
protection against radiation;
protection against external gases and fluids.

Another proposed solution involves applying to the keratin fibers a coating based on aerogel particles. The use of these aerogels in keratin fiber coating applications, however, is limited because of the particular nature of these aerogels.

The reason is that the extreme porosity of the aerogels makes them exceptionally light. Accordingly, in contrast to the conventional powders used in cosmetics, aerogel particles have a tendency, when applied in powder form—that is, without using a binder composition whose purpose is to bind the aerogel particles to one another and to the substrate to which they are to be applied—to become detached and to flutter around these substrates.

In order to overcome this drawback, one proposed solution involves forming a paste from the aerogel particles, with the use, for example, of a binder composition, in order to prevent them fluttering. This approach is unsatisfactory because, in addition to the fact that it causes the aerogel particles to lose their lightness, it usually involves combining them with fatty or polymeric products. Moreover, the presence of solvent is manifested usually in penetration of solvent into the cells of the aerogel, thereby restricting the advantage of the aerogel.

One plus point associated with the use of aerogels is that they allow the production of coatings which are relatively thick but of low weight. For cosmetic applications, however, and especially for hair coatings, the coating must not be detrimental to the natural flexibility of the substrate to which it is applied or to properties such as softness to the touch.

There is therefore a need to be able to produce coatings which are thick or fine and are able to persist and adhere to the hair, if possible without the need to employ pasting compounds, and without detriment to the properties of flexibility and softness of the hair.

The applicant has found, surprisingly, a cosmetic treatment method comprising the formation of a coating based on an aerogel composition having a low bulk density and a marked elasticity on keratin fibers, which allows the drawbacks identified above to be overcome. In this method, the aerogel composition is formed either in situ, in other words directly on the keratin fibers, or ex situ, in which case, once formed, it is applied to the keratin fibers.

The invention provides a cosmetic treatment method comprising the formation of a coating on keratin fibers, characterized in that it comprises:

1) preparing an aerogel precursor composition comprising:
at least one organic solvent selected from C₁-C₆ alkanes, C₁-C₄ alcohols, acetone, and mixtures thereof, and
at least one precursor compound containing:
   at least one atom selected from silicon, titanium, aluminum, and zirconium,
   at least one hydroxyl or alkoxy function attached directly by an oxygen atom to the atom selected from silicon, titanium, aluminum, and zirconium, and
   at least one organic group attached directly by a carbon atom to the atom selected from silicon, titanium, aluminum, and zirconium,
      the molar ratio of the precursor compounds to the solvent being not more than 1/20;
      2) removing the solvent or solvents to form an aerogel composition having a bulk density of less than or equal to 0.35 g/cm³; and
      3) applying to the keratin fibers the aerogel composition obtained from step 2).

The invention also provides a cosmetic treatment method comprising the formation of a coating on keratin fibers, characterized in that it comprises:

1) preparing an aerogel precursor composition comprising:
at least one organic solvent selected from perfluorinated or nonperfluorinated C₁-C₄ ethers, C₁-C₆ alkanes, C₁-C₄ alcohols, acetone, and mixtures thereof, and
at least one precursor compound containing:
   at least one atom selected from silicon, titanium, aluminum, and zirconium,
   at least one hydroxyl or alkoxy function attached directly by an oxygen atom to the atom selected from silicon, titanium, aluminum, and zirconium, and
   optionally an organic group attached directly by a carbon atom to the atom selected from silicon, titanium, aluminum, and zirconium,
      2) applying to the keratin fibers the aerogel precursor composition obtained from step 1); 3) removing the solvent or solvents to form an aerogel composition having a bulk density of less than or equal to 0.35 g/cm³.

In one preferred embodiment, the invention provides a cosmetic treatment method comprising the formation of a coating on keratin fibers, characterized in that it comprises:

1) preparing an aerogel precursor composition comprising:
at least one organic solvent selected from perfluorinated or nonperfluorinated C1-C4 ethers, C₁-C₆ alkanes, C₁-C₄ alcohols, acetone, and mixtures thereof, and
at least one precursor compound containing:
   at least one atom selected from silicon, titanium, aluminum, and zirconium,
   at least one hydroxyl or alkoxy function attached directly by an oxygen atom to the atom selected from silicon, titanium, aluminum, and zirconium, and
   optionally an organic group attached directly by a carbon atom to the atom selected from silicon, titanium, aluminum, and zirconium,
      2) removing the solvent or solvents to form an aerogel composition having a bulk density of less than or equal to 0.35 g/cm³;
      3) applying to the keratin fibers the aerogel composition obtained from step 2) or the aerogel precursor composition obtained from step 1).

In this preferred embodiment the ratio of the precursor compounds to the solvent is advantageously not more than 1/20.

According to the invention:
“keratin fibers” are the hair, eyebrows, eyelashes, and body hairs, and especially the hair. The present invention is directed more particularly to the hair.
“aerogel precursor composition” is a composition in sol form, comprising at least one solvent and at least one precursor compound, which is capable of forming an aerogel composition after removal of the solvent or solvents;
“aerogel composition” is a solid, inorganic or organic-inorganic material which comprises open or closed pores or cells, and has a low bulk density. The cells typically have a size of 1 nm to 10 μm.
“precursor compounds” are compounds capable of undergoing a sol-gel conversion.

A sol is defined as a stable, colloidal dispersion of solid particles within a liquid. In the sol, the solid particles react chemically with one another until a single three-dimensional entity is formed. The sol-gel transition corresponds to the passage from the liquid state (sol) to the infinitely viscous state (the gel). The gel thus formed takes the form of a porous, solid network in equilibrium with the liquid present in its pores. After the sol-gel transition, the structure of the gel continues to evolve.

The method according to the invention overcomes the drawbacks of the prior art in that the aerogel composition obtained has an extremely low bulk density, a particular porous structure, and high flexibility characteristics.

In the method of the invention, a composition referred to as a binder composition may be used in combination. The binder composition may be either directly mixed with the aerogel composition before application to the keratin fibers, or applied beforehand or afterward to the keratin fibers. The binder composition may form on the hair a film with a thickness less than the particle size of the aerogel composition, especially when the particles have a size of more than a micrometer. In this case, the binder composition does not entirely cover the particles of the aerogel composition, these particles being held solely at their base, with the aerogel particles then projecting relative to the film of binder composition. Known coating methods using micrometer-size particles suffer from problems of harshness to the touch, owing to the presence of these projecting particles. According to the invention, the use of particles having a high flexibility or elasticity, and of a binder composition, allows the particles to be held on the hair without detriment to the feel. When a finger makes contact with the flexible aerogel composition particle, it senses less harshness than with the micrometer-scale particles of the prior art.

The binder composition may be an adhesive composition or an impasting composition. The particular porous structure of the aerogel prevents the drawbacks of the prior art that are associated with the use of an impasting composition. Waxy or polymeric compounds have less tendency to migrate into the pores of the aerogel composition than with particles of the prior art, and, consequently, do not impair the lightness properties of the aerogel composition.

In order to obtain the desired cosmetic effects, all that is needed during the method is to add active cosmetic ingredients, which are then present in the coating covering the keratin fibers that is obtained by the method of the invention and that, moreover, exhibits effective adhesion, a soft-to-the-touch appearance, and optionally an excellent flexibility.

Depending on the active cosmetic ingredients selected, therefore, the method of the invention allows the formation of a coating which endows the keratin fibers with surface effects, such as color, shine, the reduction of defects, protective effects with respect to radiation, pollutants, water, or humidity, or else allows biological effects to be produced on these keratin fibers.

The cosmetic treatment method further exhibits the features described below, taken alone or in combination.

The aerogel composition is obtained from an aerogel precursor composition which has undergone a step of extraction or removal of the solvent. The removal may be accomplished by evaporation or by displacement. This is then referred to as washing of the aerogel. The washing operation must not rob the product of its aerogel structure. Accordingly, the washing that is used has little or no solubilization effect on the material of which the aerogel composition is formed.

One preferred means of removing the solvent, when the aerogel composition is formed in situ on the keratin fibers, is its removal by heating with a hairstyling hood, a hairdryer, an infrared ray dispenser, a heating iron, heated rollers or curlers, preferably comprising an electrical heating “core” and an enclosure which keeps the heat of the hair wound around the curler or roller, or any other conventional heating apparatus. Removal by heating is performed preferably at a temperature of from −30° C. to 250° C. and preferably of from room temperature to 200° C., more preferably from 50° to 200° C., and very preferably from 70 to 150° C.

The removing of the solvent may be performed by a drying step by supercritical removal of the solvent.

As an example, supercritical drying involves compressing the gel formed, in an autoclave, with CO₂, and producing conditions which go beyond the supercritical state of CO₂ (Tc˜516 K and Pc˜7.9 MPa). The solvent is removed by depressurizing and by washing with air or nitrogen under pressure (0.3 MPa, for example). The autoclave is cooled in order to finish the drying step. This procedure produces aerogel compositions with a bulk density of around 0.2 g/cm³ or less.

Other techniques are possible for removing the solvent or solvents and producing the aerogel composition. For example, a liquefied gas may be employed that forms a stable or unstable emulsion in the liquid preparation phase (microemulsified more particularly). To this end, the components are selected accordingly and, more particularly, a liquefiable gas is used which is not miscible in the solvent, and surfactants are used. The composition is held under pressure and then depressurized. At this point, the liquefied gas forms very small bubbles. Preference is given to gases which are liquefiable such that their boiling temperature is close to the temperature at which the depressurizing operation is performed. Moreover, the precursor compounds are selected such that the setting reaction takes place at the same time as the bubbles are formed.

The precursor compounds used according to the invention preferably have the following characteristics:
when the precursor compound or compounds contain an atom selected from silicon or titanium, they comprise 2, preferably 3 or 4, hydroxyl or alkoxy functions;
when the precursor compound or compounds contain an atom selected from aluminum and zirconium, they comprise one or two hydroxyl or alkoxy functions;
the precursor compound or compounds contain 0, 1, or 2 organic functions, preferably 0 or 1;
the organic function or functions may be selected from C₁-C₁₂, preferably C₁-C₆, alkyl groups, and more preferably a group comprising 1, 2, or 3 carbon atoms; C₂-C₁₂ alkenyl groups, and C₆-C₁₂ aryl groups, the alkyl, alkenyl, or aryl groups optionally comprising one or more substituents selected from a halogen atom, an OH, ═O, NH₂, SH, N₃, SCN, or COOH moiety, an ether bridge, an amine bridge, an ester bridge or an amide bridge, and a siloxane;
mention may be made, for example, of the following organic groups: methyl; ethyl; propyl; phenyl; —C₄F₉, —C₆F₁₃, and —CH═CH₂;
the organic groups are selected with particular preference from methyl and ethyl groups;
in the alkoxy function or functions —OR, preferably, R is selected from a C₁-C₁₂ alkyl, C₂-C₁₂ alkenyl, or C₆-C₁₂ aryl group, such as a methyl, ethyl, or propyl group or a phenyl group, preferably a methoxy or ethoxy group;
the precursor compounds may further comprise a group selected from —OCF₂—CHF—CF₃, —O—CF₂—CHF₂, —NH₂, —N₃, —SCN, —CH═CH₂, —NH—CH₂—CH₂—NH₂, —N— (CH₂—CH₂—NH₂)₂, —OOC(CH₃)C═CH₂, —OCH₂—C (O)CH₂, —NH—CO—N—CO—(CH₂)₅, —NH—COO—CH₃, —NH—COO—CH₂CH₃, —NH— (CH₂)₃Si (OR)₃, —S—(CH₂)₃)Si(OR)₃, and —SH.

The precursor compounds may be alkoxysilanes or alkoxytitaniums or alkoxyaluminates.

The precursor compounds are preferably silicon-based precursor compounds such as the alkoxysilanes of formula Si (R₁)ₙ(OR₂)₄₋ₙ where:
n is an integer from 0 to 3 or 1 to 3, n preferably being 0 or 1,
each R₁ may correspond to a C₁-C₁₂, preferably C₁-C₆, alkyl group, and more preferably a group comprising 1, 2, or 3 carbon atoms, a C₂-C₁₂ alkenyl group, or a C₆-C₁₂ aryl group, the alkyl, alkenyl, or aryl groups optionally comprising one or more substituents selected from a halogen atom, an OH, ═O, NH₂, SH, N₃, SCN, or COOH moiety, an ether bridge, an amine bridge, an ester bridge, or an amide bridge, and a siloxane,
R₂ is selected from a C₁-C₁₂ alkyl or C₂-C₁₂ alkenyl group and a C₆-C₁₂ aryl group, such as a methyl, ethyl, or propyl group or a phenyl group.

Alkoxysilanes suitable for the invention may include the following compounds:
tetramethoxysilane (written TMOS), tetraethoxysilane (written TEOS), tetra-n-propoxysilane,
methyltrimethoxysilane, methyltriethoxysilane, ethyltrimethoxysilane, ethyltriethoxysilane, propyltrimethoxysilane, propyltriethoxysilane, isobutyltrimethoxysilane, isobutyltriethoxysilane, octyltrimethoxysilane, octyltriethoxysilane, hexadecyltrimethoxysilane, phenyltrimethoxysilane, phenyltriethoxysilane,
3-(trimethoxysilyl)propyl methacrylate, 3-(trimethoxysilyl)propyl acrylate, 3-(trimethoxysilyl)methyl methacrylate, 3-(trimethoxysilyl)methyl acrylate, 3-(trimethoxysilyl)ethyl methacrylate, 3-(trimethoxysilyl)ethyl acrylate, 3-(trimethoxysilyl)pentyl methacrylate, 3-(trimethoxysilyl)pentyl acrylate, 3-(trimethoxysilyl)hexyl methacrylate, 3-(trimethoxysilyl)hexyl acrylate, 3-(trimethoxysilyl)butyl methacrylate, 3-(trimethoxysilyl)butyl acrylate, 3-(trimethoxysilyl)heptyl methacrylate, 3-(trimethoxysilyl)heptyl acrylate, 3-(trimethoxysilyl)octyl methacrylate, 3-(trimethoxysilyl)octyl acrylate,
3-glycidyloxypropyltrimethoxysilane, 3-glycidyloxypropyltriethoxysilane,
3-methacryloyloxypropyltrimethoxysilane, vinyltrimethoxysilane, vinyltriethoxysilane, 3-mercaptopropyltrimethoxysilane,
3-aminopropyltriethoxysilane, 3-aminopropyltrimethoxysilane, 2-amino'thyl-3-aminopropyltrimethoxysilane, N-(n-butyl)-3-aminopropyltrimethoxysilane.

Preference is given to using tetraalkoxysilanes such as tetramethoxysilane (written TMOS) and tetraethoxysilane (written TEOS), methyltrialkoxysilanes such as trimethoxymethylsilane (MTMS), propyltrialkoxysilane, (trimethoxysilyl)propyl methacrylate (TMSPMA), or 3-aminopropyltriethoxysilane (written APTES). The aerogel composition may be obtained, for example, by reaction of trimethoxymethylsilane and 3-(trimethoxysilyl)propyl methacrylate.

The precursor compounds present in the aerosol precursor composition may be in free (monomeric) form or in prepolymerized form (where a certain number of monomers have reacted before application to the hair). The prepolymerized precursors include those obtained from a substoichiometric hydrolysis of the TEOS under acid catalysis. The advantage of using precursors in prepolymer form is that it enables a reduction to be made in the gelling time, since the hydrolysis and condensation reactions are already at a significantly advanced state. It is therefore possible to use, for example, a prehydrolyzed ethyl orthosilicate solution Silbond® H-5.

The aerogel precursor composition may comprise the following characteristics:
the precursor compounds and the solvent are present in the aerogel precursor composition in a molar ratio of the precursor compounds to the solvent of not more than 1/20;
the aerogel precursor composition comprises at least 10% by weight, relative to the total weight of the precursor compounds, of precursor compounds containing:
   at least one atom selected from silicon, titanium, aluminum, and zirconium,
   at least one hydroxyl or alkoxy function attached directly by an oxygen atom to the atom selected from silicon, titanium, aluminum, and zirconium, and optionally
   at least one organic group attached directly by a carbon atom to the atom selected from silicon, titanium, aluminum, and zirconium.

Other ingredients may be introduced into the aerogel precursor composition, these ingredients being intended for modification of the mechanical, absorption, surface tension, permeability, color, and/or surface condition properties. According to the invention, these compounds are called coingredients. They are also intended to enhance or limit the adhesion to the substrate. The aerogel precursor composition may comprise at least one coingredient selected from activated carbon as powder. Lastly, it is possible to use coingredients capable of reacting with the aerogel composition. For example,
a polymerization or crosslinking, with the aid of compounds such as:
   free monomers or reactive groups grafted onto oligomers or polymers, capable of entering into a radical or ionic polymerization reaction, such as those of acrylate or cyanoacrylate monomers;
   free monomers or reactive groups grafted onto oligomers or polymers, capable of reacting by condensation, such as, for example, sol-gel monomers (alkoxysilane, alkoxytitanium, aluminum salts, etc.), silanol-functional polymers, or else crosslinkers such as diisocyanates and dithiols;
physical crosslinking such as that obtained by the action of a cationic salt (generally dicationic) with certain compounds, such as acrylic polymers or cellulosic gels.

The skilled person will select these coingredients, ensuring that their presence in the aerogel precursor compositions of the present invention is not harmful to the inherent advantageous properties of these compositions, in particularly in the dispersed state, or to the reactivity of the precursors during the drying of the cosmetic coating.

When the aerogel composition is formed ex situ, it takes the form of particles in the form of grains, flakes, or powder. By “powder” is meant individualized particles. By “flakes” are meant flat particles. By “grain” is meant a more or less agglomerated assembly of particles. The particles preferably have a size of at least 0.1 μm, preferably of at least 1 μm, and more preferably of between 2 and 100 μm. By “size” of the particles is meant the average dimension of the particles. The size of the particles is evaluated by means of observation under an electron microscope, following treatment of the sample with a gold plasma of a few nanometers.

The aerogel composition comprises pores having a size between 1 nm and 10 μm, preferably, 50% of the pores are less than 40 nm, and 90% of the pores are less than 100 nm; by “size” of the pores is meant the average dimension of the pores.

The size of the pores is evaluated by means of observation under an electron microscope, following treatment of the sample with a gold plasma of a few nanometers. In order to evaluate the pore size in the smallest sizes, the method of mercury porosity may be used. The measurements may be conducted with a Carlo-Erba 2000 porosimeter. The pore size distribution may be obtained by the Washburn equations (E. W. Washburn, Proc. Natl. Acad. Sci. USA 7 (1921) 115.), if the mercury is incorporated into the pores, or by the theory developed by Pirard and coworkers (called Buckling theory), when the mercury does not penetrate the network (R. Pirard, S. Blacher, F. Brouers, J. P. Pirard, J. Mater, Res. 10 (1995) 2114. and R. Pirard, A. Rigacci, J. C. Maréchal, D. Quenard, Br. Chevalier, P. Achard, J. -P. Pirard, Polymer 44 (2003) 4881).

The aerogel composition obtained by the method of the invention may be obtained from an aerogel precursor composition comprising:
at least one precursor compound containing:
   at least one atom selected from silicon, titanium, aluminum, and zirconium,
   at least one hydroxyl or alkoxy function attached directly by an oxygen atom to the atom selected from silicon, titanium, aluminum, and zirconium, and optionally
   at least one organic group attached directly by a carbon atom to the atom selected from silicon, titanium, aluminum, and zirconium,
activated carbon as powder,
   said aerogel composition having a bulk density of less than or equal to 0.35 g/cm³, preferably less than or equal to 0.25 g/cm³, and more preferably less than or equal to 0.05 g/cm³.

In the aerogel composition comprising an activated carbon powder, the activated carbon powder preferably represents at least 0.1% by weight, relative to the total of the aerogel composition, or from 0.4% to 80%, preferably 2% to 60%, more preferably from 10% to 60%, and more preferably still from 15% to 50% by weight of activated carbon powder.

The addition of an increasing amount of activated carbon powder makes it possible, surprisingly, to lower the bulk density of the aerogel composition while also improving the flexibility properties. The addition of the activated carbon powder as described in example 1 below significantly enhances the flexibility and lightness of the aerogel composition.

According to another embodiment, the method of the invention involves a transfer device. In this case, the method further comprises:
applying the aerogel precursor composition to a transfer device,
removing the solvent or solvents, to form an aerogel composition on the transfer device,
applying the transfer device coated with the aerogel composition to the keratinous materials.

According to the invention, a “transfer device” is a substrate on which the aerogel composition is produced with the objective of subsequently applying said composition to the keratin fibers by applying the coated transfer device; the transfer devices which can be used according to the invention are for example, a patch, a comb, a hairbrush, or rollers.

According to one embodiment, the aerogel composition may have a notable elasticity. These properties may in particular be obtained by using an aerogel precursor composition exhibiting a particular dilution. In this case, the precursor compounds and the solvent are present in the aerogel precursor composition in a molar ratio of the precursor compounds to the solvent of not more than 1/20, preferably not more than 1/30. The substantial dilution produces the required flexibility properties.

The elasticity of a material is evaluated by a variety of criteria. One is the resistance to the force applied. The modulus of elasticity expresses this resistance in the primary effects of the force. The material in this case is not deformed very much. The aerogel according to the invention is required to exhibit a moderate modulus of elasticity. It will be noted that the measurement of the modulus of elasticity requires techniques appropriate to the porous or granular character. The measurements may be affected by error or underestimation, as indicated by M. Moner-Girona et al. In the article Applied. Physics. A 74, 119-122 (2002). Accordingly, among techniques such as the measurement of speed of ultrasound through a material, AFM in contact mode, and Knoop indentation, the microindentation technique described hereinafter will be used.

The elasticity of a material is also evaluated by its ability to undergo a deformation and regain a substantial part of its initial form: recovery. Here again, microindentation is used. This method is described in the article by M. Moner-Girona et al. In the article Applied. Physics. A 74, 119-122 (2002), “Micromechanical properties of carbon-silica aerogel composites”.

A Nanotest 550 instrument from the British commercial company Micromaterials is used. A “Berkovich” triangular pyramidal-based indenter is employed. The measurements are made on a test specimen a few millimeters in thickness. During the compression “P”, the system measures the penetration “h”. Measurement is made at 0.05 mM/s during compression and decompression. The compression is determined so as to produce a penetration h of approximately 5 μm. Ten cycles are carried out at different sites on the test specimen in order to form an average.

The modulus of elasticity is evaluated using the following formula:

$$\frac{d P}{d h} = \frac{2 E}{\sqrt{π} ( 1 - {v}^{2} )} \sqrt{A}$$

where:
dP/dh is the slope of the origin of the decompression curve,
E is the modulus of elasticity,
v is the Poisson's modulus (0.2)
and A is the projected area of the indenter.

The recovery is evaluated by the following formula:

$$EP = \frac{{h}_{1} - {h}_{0}}{{h}_{1}}$$

where:
ht is the maximum depth
hp is the depth not recovered after decompression.

For example, if a material is highly elastic, with hp being close to zero, the recovery EP will be close to 1. A material without elasticity would have a low recovery.

According to the invention, the aerogel composition preferably has a recovery of at least 20%, preferably at least 40%, more preferably at least 50%, and very preferably at least 60%.

The modulus of elasticity is preferably less than 500 MPa, preferably less than 100 MPa, more preferably less than 50 MPa, and very preferably less than 10 MPa.

The aerogel compositions are produced from a sol, subsequently forming gels which are two-phase solid-liquid systems composed of an interpenetrating twin network which is continuous and three-dimensional, one being solid and the other liquid. The aerogel compositions are obtained by extraction and substitution of the solvent phase by air, with very little or no densification of the solid network. The extraction or drying phase therefore involves extracting the solvent present in the pores of the gel while attempting to retain as effectively as possible the structure and the integrity of the solid network formed in the gel step.

In one embodiment of the invention, the aerogel precursor composition in sol form is extremely diluted, and this is manifested in a molar ratio of the precursor compounds to the solvent of not more than 1/20, preferably not more than 1/30. The substantial dilution produces the required flexibility properties.

The process for preparing aerogel precursor compositions based on these compounds is described in the following article: Synthesis of flexible silica aerogels using methyltrimethoxysilane (MTMS) precursor, by VENKATESWARA RAO A., BHAGAT Sharad D., HIRASHIMA Hiroshi; PAJONK G. M., “Journal of colloid and interface science” ISSN 0021-9797 CODEN JCISA5, 2006, vol. 300, No. 1, pp. 279-285De A. This article sets out a process for preparing an aerogel prepared from MTMS (H3C—Si—(OCH₃)₃)

The aerogel precursor composition is prepared by first mixing MTMS, methanol, and water in the presence of acid (oxalic acid) and then a base (ammonium hydroxide). Following ageing, a supercritical drying operation is performed, leading to the production of a flexible aerogel at the time of depressurizing.

The production of a flexible aerogel is promoted by the use of a diluted composition.

The aerogel composition may further comprise an organic aerogel which may be selected, for example, from organic aerogels based on polyisocyanate chemistry, such as those described in patent EP 0 710 262, or aerogels formed by reaction of polyisocyanates and polyols.

Preferred organic aerogels are those obtained by reaction of polyol and polyisocyanate monomers. The polyisocyanates may be selected from aliphatic, cycloaliphatic, and aromatic polyisocyanates. A particular example is diphenylmethane diisocyanate (MDI) in the form of its 2,4′, 2,2′, and 4,4′ isomers (pure MDI) and mixtures of these isomers, which are known in the art under the name “crude” MDI. The polyol may be selected from ethylene glycol, propylene glycol, glycerol, trimethylolpropane, triethanolamine, penta-erythritol, sorbitol, and sucrose. Consequently the aerogel may be obtained by reaction of compounds comprising at least two isocyanate functions with optionally at least one polyol, the polyol being preferably a sucrose.

The organic aerogel is preferably obtained by reaction of precursors such as MDI and sucrose. For example, it is possible to use those described in the following article: “Preparation of polyurethane-based aerogels and xerogels for thermal superinsulation”, A. Rigacci, J. C. Marechal, M. Repoux, M. Moreno and P. Achard; Journal of Non-Crystalline Solids, 350 (2004) 372-378.

According to this preparation process, a polyol (sucrose for example) and an MDI diisocyanate (4,4′-methylenebis(phenyl isocyanate), for example) are dissolved in a solvent which is able to dissolve the two monomers and the polymer which is to form (in the present context, the solvent consists of a mixture of dimethyl sulfoxide, DMSO, and ethyl acetate). A diamine catalyst (for example, 1,4-diazobicyclo[2.2.2]octane, DABCO) is then added at ordinary temperature, thereby causing gelling to take place. This is followed by a drying step by supercritical removal of the solvent.

Lastly, the aerogel composition may be functionalized, either by the presence of free reactive functions, or by aftertreatment.

The aerogel precursor composition may be obtained either by introducing the soluble forms of the precursor compounds directly into the solvent phase, the solvent phase consisting of a solvent or solvents, or by mixing solutions prepared beforehand.

The solvent phase must preferably be such that the soluble forms of the various chemical elements required in order to obtain the precursor are soluble in said solvent or else capable of forming homogeneous and stable colloidal solutions. According to the method of the invention, the aerogel precursor composition comprises at least one organic solvent selected from acetone, C₁-C₄ alcohols, C₁-C₆ alkanes, and mixtures thereof. The precursor composition further comprises water, the water being necessary in order to initiate the hydrolysis and condensation reactions that allow the gel, and then the aerogel composition, to form. The C₁-C₆ alkanes may be selected from butane, such as isobutane, or pentane, such as isopentane.

Preference is given to using two types of solvents, the solvents being immiscible, with one having a fairly low boiling temperature relative to the other. Accordingly, it is possible to use water and isopentane. Since the two solvents are immiscible, a stable or unstable emulsion may be formed by stirring and/or by stabilizers such as surfactants.

The composition may include a catalyst. This catalyst may be added directly to the precursor mixture, or added separately via a pretreatment or an aftertreatment following the application of the pre-aerogel composition.

The aerogel compositions obtained in the method of the invention are different from the expanded spheres sold, for example, under the name Expansel®, in that the aerogel compositions are composed of particles studded with holes, whereas the porous spheres owe their low bulk density to their formation from a shell (100 nm, for example) and from a hollow zone (40 μm, for example). In both cases, the bulk density is low, and for the expanded spheres as well may range below 0.25 g/cm³.

According to the invention, the solid material is sufficiently deformable for the aerogel to be flexible. However, the solid material is sufficiently solid for the aerogel composition not to collapse in on itself. Accordingly, the aerogel compositions should not be confused with a simple foam obtained by expansion of a gas in a liquid. The aerogel compositions retain their porous form over time, whereas the foams, of the kind produced by expanding an emulsion, eventually collapse and lose their density. Moreover, the aerogel compositions are the result of a chemical reaction (crosslinking in general).

Only the aerogel compositions having a bulk density of less than or equal to 0.35 g/cm³ are suitable for implementing the method of the invention, with preferred compositions being those that also have a modulus (E) of less than 0.5 GPa. For example, the aerogel compositions obtained conventionally by sol-gel synthesis and alkaline catalysis do not exhibit the required flexibility. They are obtained by expansion and then optionally undergo heating at high temperature (900° C., for example). The measurement of the hardness of the material by nanoindentation shows that moduli (E) of typically from 3.8 GPa to 1.2 GPa are obtained. With such hardnesses, the materials have no possibility of being deformed reversibly (cf. studies by LUCAS Erik M., DOESCHER Michael S., EBENSTEIN Donna M., WAHL Kathryn J., ROLISON Debra R., Journal of non-crystalline solids ISSN 0022-3093 CODEN JNCSBJ ISA-7: International Symposium on Aerogels #7, Alexandria Va., UNITED STATES (Feb. 11, 2003) 2004, vol. 350 (415 p.) [document: 9 p.] (41ref.) pp. 244-252).

According to the invention, the aerogel compositions used can be much more flexible. The moduli of elasticity E are typically less than 0.5 GPa and preferably less than 100 MPa.

When they are subjected to a mechanical stress, contraction or extension, the aerogel compositions according to the invention deform and regain a substantial part of their initial form when the mechanical stress is discontinued. Their recovery is greater than 20%.

The aerogel compositions of the invention have little or no ability to absorb moisture or to absorb and transmit moisture.

In order to check the absorption of moisture, one known test involves following the change in the weight of the materials in an atmosphere with a relative humidity (RH) of 65% at ordinary temperature. The material preferably does not absorb water. Accordingly, after contact for one day, the aerogel has a level of water absorption in an atmosphere of 65% relative humidity at ordinary temperature of less than 5% by mass. This point is important so that the coating does not absorb the perspiration (or exogenous water) and so lose its property of lightness.

According to another embodiment of the invention, the aerogel composition may be combined with a cosmetic composition. In this case, the aerogel composition represents at least 50%, preferably at least 80%, and more preferably 90% by volume, relative to the total volume of the aerogel composition and of the cosmetic composition (combination of the two compositions).

According to the invention, when the aerogel composition is formed ex situ, an “aerogel composition combined with a cosmetic composition” means alternatively:

a) the mixing of the two compositions prior to application to the keratin fibers;
b) the prior application of the aerogel composition to the keratin fibers, followed by the application of the cosmetic composition; in this case, the cosmetic composition is added to the aerogel composition after application of said composition to the keratin fibers;
c) the application of the cosmetic composition followed by the application of the aerogel composition.

According to the invention, when the aerogel composition is formed in situ, an “aerogel composition combined with a cosmetic composition” means alternatively:

a) the prior application of the aerogel precursor composition, leading, after removal of the solvent, to the aerogel composition, followed by the application of the cosmetic composition; in this case, the cosmetic composition is added to the aerogel composition after application of said composition to the keratinous materials;
b) the application of the cosmetic composition followed by the application of the aerogel precursor composition.

The cosmetic composition may comprise at least one complementary ingredient. The complementary ingredient is preferably selected to facilitate the application of the aerogel composition, and is preferably a solvent, characterized in that the aerogel composition is not soluble therein, or rheological agents such as thickeners. Other complementary ingredients that may be mentioned include “hold” ingredients for enhancing the hold of the particles, such as polymers (polyacrylic polymers, polyacrylates, polyesters, polyamides, etc.), which are reactive or unreactive, and monomers or oligomers, waxes, oils, and hot-melt compounds. These ingredients are more particularly reactive. They may be reactive with themselves and/or with respect to the functions of the aerogel composition. In the latter case, it is possible to use silanol functions in order to form a reaction between the aerogel composition and the hold ingredient.

The complementary ingredients may therefore be selected from solvents, liquids, compounds in solution, dyes, pigments, dihydroxyacetone (DHA), fragrances, gases such as NO, active biological ingredients, and filters. The complementary ingredients may be contained in the porous structure of the aerogel composition.

The aerogel composition may serve if required as a reservoir for a solvent, a liquid, or a compound in solution. In that way the ingredient is carried. It may evaporate or escape to reach the hair, or may spread into the atmosphere.

The complementary ingredients may be contained in the porous structure of the aerogel composition or in the material of the aerogel (i.e., in the material forming the walls of the cells, or are grafted to the material of the aerogel composition). The complementary ingredients may typically form up to 40% by volume of the aerogel composition.

Hence the aerogel composition may be applied with a dye or a filter as a complementary ingredient. In this way, quantities of product may be placed without the ingredient coming directly into contact with the hair.

The cosmetic composition may also be selected from an impasting composition or an adhesive composition, comprising or not comprising said complementary ingredients.

The use of an impasting composition is not necessary. However, the combination of an aerogel composition and an impasting composition allows:
the particles to be adhered to the hair;
the particles to be bonded to one another;
the particles to be bonded to other compounds, such as dye compounds and pigments.

The impasting composition is selected so as not fundamentally to modify the aerogel composition, which must retain its cellular appearance. Part of the impasting composition may be infiltrated into the cells. This point is verified by looking at the material by microscopy. The impasting composition may soften the walls of the cells, but the cells must be preserved.

In addition to the solvents, the impasting composition contains materials which are capable after drying of giving a flexible, soft and/or tacky film, such as natural or synthetic polymers, especially modified or unmodified polysaccharides such as guar gum, carrageenan gum, hyaluronic acid, proteins such as keratin hydrolysates, for example, synthetic polymers such as acrylate or acrylic or methacrylate or methacrylic copolymers, polyesters, and polyurethanes. Depending on the selection of these polymers, a soft, tacky or rigid or elastomeric film is obtained.

Where an impasting composition and a flexible aerogel composition are employed, it is apparent that the particles of the aerogel composition are much more resistant than a simple inflexible aerogel. This is the case, in particular, when the aerogel particles have a size of greater than 1 μm, for two reasons.

On the one hand, it is extremely difficult to cause micrometer-size particles to hold to the hair, since they are pulled off by friction. The flexible aerogel composition particles are more resistant to friction, and the effect of the impasting composition is therefore more efficacious.

Furthermore, the known coating methods using micrometer-size particles present problems of roughness to the touch, owing to the presence of these particles projecting from the film of impasting composition, since the micrometric particles generally have a size larger than the thickness of the film applied to the keratin fibers.

According to the invention, when a coating is formed that comprises the aerogel composition and an impasting composition, the particles of the aerogel composition are retained on the hair and, when a finger comes into contact with the particle of flexible aerogel composition, it senses less roughness than with the micrometric particles of the prior art.

An impasting composition may be used that employs a chemical or physicochemical reaction. For example,
a polymerization or crosslinking by means of compounds such as:
   free monomers or reactive groups grafted onto oligomers or polymers, capable of entering into a radical or ionic polymerization reaction, such as those of acrylate or cyanoacrylate monomers;
   free monomers or reactive groups grafted onto oligomers or polymers, capable of reacting by condensation, such as, for example, sol-gel monomers (alkoxysilane, alkoxytitanium, aluminum salts, etc.), silanol-functional polymers, or else crosslinkers such as diisocyanates and dithiols;
physical crosslinking such as that obtained by the action of a cationic salt (generally dicationic) with certain compounds, such as acrylic polymers or cellulosic gels.

The adhesive composition may be a composition of the kind referred to as pressure-sensitive adhesive, denoted by the acronym PSA. These compositions generally comprise a film-forming compound and a solvent. When these compositions are applied to a substrate, a film forms by evaporation or absorption of solvent into the substrate (after a few minutes). This film has significant and long-lasting adhesion properties. These particular adhesion properties mean, when an article is contacted with the film, that gentle pressure is sufficient to bring about adhesion of the article with the surface of the film. This adhesion is characterized by the need to produce a force in order to detach the article from the surface.

The pressure-sensitive adhesive composition may lose some or all of its adhesive properties in the presence of the aerogel composition. The pressure-sensitive adhesive composition is therefore defined by its adhesive property in the absence of aerogel composition.

An example of an adhesive composition suitable for the invention is a composition comprising at least one solvent, preferably selected from water and ethanol, and at least one film-forming compound (active substance), preferably selected from sulfonic polyesters. The film-forming compound represents preferably 1% to 20%, more preferably from 4% to 10% by weight of the adhesive composition. The sulfonic polyesters which can be used in the method of the invention are sold for example by EASTMAN. Preferred among these polymers are those sold under the names AQ 1045®, AQ 1350®, and AQ 14000®. A particularly preferred commercial product is the product sold under the name AQ 1350® by EASTMAN Chemicals.

The combination of the aerogel composition and the adhesive composition forms a cosmetic composition.

The invention likewise provides a cosmetic composition comprising by volume, relative to the total volume of the cosmetic composition:
5% to 30%, preferably 10% to 15% of aerogel composition having a bulk density of less than or equal to 0.35 g/cm³, obtained from an aerogel precursor composition comprising at least one precursor compound containing:
   at least one atom selected from silicon, titanium, aluminum, and zirconium,
   at least one hydroxyl or alkoxy function attached directly by an oxygen atom to the atom selected from silicon, titanium, aluminum, and zirconium, and optionally
   at least one organic group attached directly by a carbon atom to the atom selected from silicon, titanium, aluminum, and zirconium;
70% to 95%, preferably 85% to 90% of adhesive composition.

The coating serves as a coating for protecting the skin or hair, without making them heavy, as follows:
protection against thermal differences;
protection against radiation, such as UV or IR;
protection against wind, pollutants, or pollens;
protection against friction.

Lastly, the treatment method of the invention is especially suitable as a method for treating keratin fibers to protect said keratin fibers against heat or cold, radiation such as UV or IR, wind, pollutants, pollens, and friction.

The coating method of the invention is especially suitable as a method for treating keratinous materials to obtain surface effects, such as color, shine, reduction of defects, protective effects with regard to radiation, pollutants, water, or humidity, or for producing biological effects.

The cosmetic treatment method of the invention preferably comprises the formation of a coating with a thickness of between 10 nm and 100 μm.

Lastly, the coating method of the invention is also suitable as a method for treating keratinous materials in order to absorb body fluids.

When the aerogel composition is produced either ex situ and on a transfer device or in situ on the keratin fibers, and when the solvent is subsequently removed, a number of other techniques for removing the solvent are possible.

According to one embodiment, following the step of applying the aerogel precursor composition to the keratinous materials or to the transfer device, a step of injecting gas under pressure by means of a suitable apparatus is practiced. Typically this apparatus is a perforated surface (a grid), or a surface equipped with a multiplicity of pressurized gas injection points. This application is carried out with the application of a pressure during the injection time, so that the injected bubbles remain “blocked” in the material without being able to release from it.

According to another embodiment, a compound that releases a gas is added to the formula, such as a carbonate, said compound being applied before or after the step of applying the aerogel precursor composition. The carbonate, at the time when the aerogel precursor composition is applied, liberates CO₂.

Lastly, according to another embodiment, a pressurized composition is applied which will undergo degassing and form bubbles. Typically this is an aerogel precursor composition which is pressurized at or close to supercritical conditions and is applied at the time of depressurizing or just after depressurizing.

When these three latter embodiments are carried out after the step of applying the aerogel precursor composition to the keratinous materials, it is necessary with preference to use a chamber which is suitable for enclosing the hair. This is typically a roller onto which the hair is wound. The core of the roller is heated. The core of the roller may comprise, for example, a resistance heater (supplied and regulated) or a reservoir possibly containing a heat transfer fluid. Moreover, a cylindrical casing, generally made of rigid plastic, may be fitted to the rollers. This casing surrounds the hair wound onto the roller. This casing contains a slot through which the hair is introduced. The slot may be equipped with a system of two lips, made of rubber, for example, to provide for complete or partial sealing. The two lips may separate to facilitate the passage of the hair on installation and then close to ensure sealing. The casing may be a thermal insulator.

The examples which follow illustrate the present invention.

### EXAMPLE 1

Synthesis takes place as described in the article by M. Moner-Girona et al, “Micromechanical properties of carbon-silica aerogel composites” appearing in the journal Applied. Physics. A 74, 119-122 (2002). The silica sols are prepared by dissolving methyltrimethoxysilane in acetone and adding water during mixing. The molar ratios are given in the article.

Activated carbon powder (Norit-Darco KB) is added in various ratios at the time when the sols undergo gelling. The carbon powder is activated by steam treatment at 1000° C. The particles are aggregated and form objects of several microns. Supercritical extraction of the solvent is performed. CO₂ is injected at 200 bar, followed by heating at 280° C. Depressurization is carried out in order to withdraw the solvent and the CO₂.

By this method, four aerogel compositions are produced. They have different levels of carbon particles, affecting their characteristics.

The first aerogel composition contains no carbon particles. It has a bulk density of 0.24, a modulus of 190 MPa, and a low recovery of 6%.

The second aerogel composition contains 2% by weight of carbon particles. It has a bulk density of 0.21, a modulus of 52 MPa, and a recovery of 29%.

The third aerogel composition contains 15% by weight of carbon particles. It has a bulk density of 0.17, a modulus of 28 MPa, and a recovery of 45%.

The fourth aerogel composition contains 50% by weight of carbon particles. It has a bulk density of 0.15, a modulus of 23 MPa, and a recovery of 54%.

The aerogel compositions are then ground into fine particles.

The four powders of aerogel composition that are obtained are introduced respectively at 12% (by volume) into a PSA AQ 1350® adhesive composition (Eastman Chemicals) at 5% by weight (based on active substance). The formulas are subsequently applied to the hair (1 g/g of hair).

The first formula gives a rough, powdery outcome. When subjected to friction (combing for the hair), the coating exhibits poor persistence over time.

Better performances are obtained with the second, third, and fourth formulas. The fourth formula gives the best results in terms of softness on the hair.

### EXAMPLE 2

The synthesis takes place as described in the article “Synthesis of flexible silica aerogels using methyltrimethoxysilane (MTMS) precursor”, A. Venkateswara Rao, Sharad D. Bhagat, Hiroshi Hirashima, G. M. Pajonk; Journal of Colloid and Interface Science, ISSN0021-9797 CODEN JCISA5, 2006, vol. 300, No. 1, 279-285.

Synthesis is performed by a two-step sol-gel reaction, followed by drying under supercritical conditions.

Methyltrimethoxysilane (MTMS, H₃C—Si—(OCH₃)₃) is used, and is dissolved in a methanol/water mixture and a first acid catalyst (oxalic acid). Then a second basic catalyst (ammonium hydroxide) in water is added dropwise. After contact with a first catalyst (oxalic acid) for 30 minutes. The molar ratios are as follows:
H₂O/MTMS=8
MeOH/MTMS=35.

Methanol is then added and the gel is left to stand for 2 days. The aerogel is obtained by supercritical drying. For this purpose the gel is placed in a 600 ml autoclave with addition of 100 ml of methanol. By heating at 538 K and holding the pressure at 1 MPa, the methanol is evacuated in gas form. After cooling and removal of the last methanol vapors, the aerogel is recovered.

An aerogel is obtained which has a bulk density of 0.05 g/cm³. Its recovery is greater than 80%. Its modulus of elasticity is less than 1 MPa.

This aerogel is ground.

The resulting powder, corresponding to the aerogel composition of the invention, may be applied directly to keratin fibers.

The powder may be applied to hair which has been covered beforehand with a PSA adhesive (AQ 1350®, for example).

The powder may be mixed with a cosmetic composition comprising a complementary ingredient intended to enhance its hold on the skin or the hair, such as a polymer in solution or a gel or a cream.

### EXAMPLE 3

1. Preparation of a composition formed of 50 ml of TEOS and 40 ml of pure ethanol.
2. Preparation of a composition containing 35 ml of pure ethanol, 70 ml of water, 0.275 ml of 30% aqueous ammonia, and 1.21 ml of 0.5 M ammonium fluoride.
3. The two preparations are mixed together to give the aerogel precursor composition. This composition is left to stand.

The resulting aerogel precursor composition is applied to dry hair, and the hair is separated with a comb.

After an hour, the hair is heated at 80° C. with a current of air.

The hair thus coated with a nascent aerogel continues to evolve to give, within the hours which follow, a coating that provides a styling and texturing effect.

### EXAMPLE 4

The following mixtures are prepared:

1. Preparation of a composition formed of 50 ml of Silbond H-5 and 50 ml of pure ethanol.
2. Preparation of a composition containing 35 ml of pure ethanol, 75 ml of water, and 0.35 ml of 30% aqueous ammonia.
3. The two preparations are mixed together to give the aerogel precursor composition. This composition is left to stand.

The whole is applied to dry hair, and the hair is separated with a comb.

After an hour, the hair is heated at 80° C. with a current of air.

The hair thus coated with a nascent aerogel continues to evolve to give, within the hours which follow, an aerogel coating.

1-18. (canceled)

19. A cosmetic treatment method comprising the formation of a coating on keratin fibers, the method comprising:
1) preparing an aerogel precursor composition comprising:
at least one organic solvent chosen from perfluorinated or nonperfluorinated C₁-C₄ ethers, C₁-C₆ alkanes, C₁-C₄ alcohols, acetone, and mixtures thereof, and
at least one precursor compound containing:
at least one atom chosen from silicon, titanium, aluminum, and zirconium,
at least one hydroxyl or alkoxy functional group attached directly by an oxygen atom to the atom chosen from silicon, titanium, aluminum, and zirconium, and
optionally an organic group attached directly by a carbon atom to the atom chosen from silicon, titanium, aluminum, and zirconium,
wherein the molar ratio of the precursor compounds to the solvent is not more than about 1:20;
2) removing the at least one solvent to form an aerogel composition having a bulk density of less than or equal to about 0.35 g/cm³; and
3) applying to the keratin fibers the aerogel composition obtained from step 2) or the aerogel precursor composition obtained from step 1).

20. The cosmetic treatment method according to claim 19, wherein the aerogel composition has a recovery of at least about 20%.

21. The cosmetic treatment method according to claim 19, wherein the aerogel composition has a modulus of elasticity, E, of less than about 500 MPa.

22. The cosmetic treatment method according to claim 19, wherein the removing the solvent is performed by a drying step by supercritical removal of the solvent.

23. The cosmetic treatment method according to claim 19, wherein the removing the solvent is performed by heating with a hairstyling hood, a hairdryer, an infrared ray dispenser, a heating iron, or heated rollers.

24. The cosmetic treatment method according to claim 19, wherein the at least one precursor compound is chosen from alkoxysilanes of formula:
Si(R₁)ₙ(OR₂)₄₋ₙ
where:
n is an integer from 0 to 3,
R₁ is chosen from C₁-C₁₂ alkyl groups, C₂-C₁₂ alkenyl groups, and a C₆-0₁₂ aryl groups, the alkyl, alkenyl, or aryl groups optionally comprising one or more moieties chosen from halogen atoms, an —OH, ═O, —NH₂, —SH, —N₃, —SCN, and —COOH moiety, an ether bridge, an amine bridge, an ester bridge, or an amide bridge, and a siloxane;
R₂ is chosen from C₂-C₁₂ alkyl groups, C₂-C₁₂ alkenyl groups, and C₆-C₁₂ groups.

25. The cosmetic treatment method according to claim 19, wherein the at least one precursor compound is chosen from tetramethoxysilane, tetraethoxysilane, and tetra-n-propoxysilane.

26. The cosmetic treatment method according to claim 19, wherein the at least one precursor compound is in free form or in prepolymerized form.

27. The cosmetic treatment method according to claim 19, wherein the aerogel precursor composition comprises at least about 10% by weight, relative to the total weight of the precursor compounds, of at least one precursor compound containing:
at least one atom chosen from silicon, titanium, aluminum, and zirconium,
at least one hydroxyl or alkoxy functional group attached directly by an oxygen atom to the atom chosen from silicon, titanium, aluminum, and zirconium, and
optionally at least one organic group attached directly by a carbon atom to the atom chosen from silicon, titanium, aluminum, and zirconium.

28. The cosmetic treatment method according to claim 19, wherein the aerogel composition comprises pores having a size ranging from about 1 nm to about 10 μm.

29. The cosmetic treatment method according to claim 19, wherein the aerogel precursor composition comprises activated carbon powder.

30. The cosmetic treatment method according to claim 19, wherein the aerogel composition is in the form of particles in grain, flake, or powder form.

31. The cosmetic treatment method according to claim 19, wherein the aerogel composition is combined with a cosmetic composition.

32. The cosmetic treatment method as claimed in claim 31, wherein the cosmetic composition is selected from an impasting composition or an adhesive composition.

33. The cosmetic treatment method according to claim 19, wherein the aerogel composition obtained from step 2) or the aerogel precursor composition obtained from step 1) is applied to the keratin fibers in step 3) to form a coating having a thickness ranging from about 10 nm to about 100 μm on the keratin fibers.

34. An aerogel composition obtained from an aerogel precursor composition comprising:
(a) at least one precursor compound containing:
at least one atom chosen from silicon, titanium, aluminum, and zirconium,
at least one hydroxyl or alkoxy functional group attached directly by an oxygen atom to the atom chosen from silicon, titanium, aluminum, and zirconium, and
optionally at least one organic group attached directly by a carbon atom to the atom chosen from silicon, titanium, aluminum, and zirconium;
and
(b) activated carbon powder,
wherein said aerogel composition has a bulk density of less than or equal to about 0.35 g/cm³.

35. The aerogel composition according to claim 34, wherein the activated carbon powder is present in the composition in an amount of at least about 0.1% by weight, relative to the total weight of the aerogel composition.

36. A cosmetic composition comprising by volume, relative to the total volume of the cosmetic composition:
(a) about 70% to about 95% of adhesive composition, and
(b) about 5% to about 30% of aerogel composition having a bulk density of less than or equal to about 0.35 g/cm³, obtained from an aerogel precursor composition comprising at least one precursor compound containing:
at least one atom chosen from silicon, titanium, aluminum, and zirconium,
at least one hydroxyl or alkoxy functional group attached directly by an oxygen atom to the atom chosen from silicon, titanium, aluminum, and zirconium, and
optionally at least one organic group attached directly by a carbon atom to the atom chosen from silicon, titanium, aluminum, and zirconium.

37. The cosmetic treatment method according to claim 24, wherein n is 0 or 1.
